(19) United States
(12) Patent Application Publication
(10) Pub. No.: US 2022/0072089 A1
(43) Pub. Date: Mar. 10, 2022

(54) CANCER THERAPEUTIC COMPOSITIONS AND METHODS

(71) Applicant: University of Vermont and State Agricultural College, Burlington, VT (US)

(72) Inventors: Brian CUNNIFF, Burlington, VT (US); Nicholas HEINTZ, Burlington, VT (US); Terri MESSIER, Burlington, VT (US)

(73) Assignee: University of Vermont and State Agricultural College, Burlington, VT (US)

(21) Appl. No.: 17/415,772

(22) PCT Filed: Dec. 19, 2019
(86) PCT No.: PCT/US2019/067494

§ 371 Date: Jun. 18, 2021

Related U.S. Application Data
(60) Provisional application No. 62/781,673, filed on Dec. 19, 2018.

(51) Int. Cl. A61K 38/12; A61K 31/14; A61K 38/06; A61K 45/06; A61P 35/00

(52) U.S. Cl. CPC A61K 38/12; A61K 31/14; A61P 35/00; A61K 45/06; A61K 38/063

(57) ABSTRACT

The invention relates, in part, to compounds, compositions, and methods comprising Thiostreopton (TS) that are useful to treat cancer in cells and subjects.

### RELATED APPLICATIONS

This application claims benefit under 35 U.S.C. §119(e) of U.S. Provisional application Ser. No. 62/781,673 filed Dec. 19, 2018, the disclosure of which is incorporated by reference herein in its entirety.

### FIELD OF THE INVENTION

The invention relates, in part, to compositions and methods for treating cancer, including but not limited to malignant mesothelioma.

### BACKGROUND OF THE INVENTION

Treatment for various cancers may include administration of chemotherapeutic agents to kill fast-dividing cells in a subject with the cancer. Although there are a number of chemotherapeutic agents that are administered to treat cancers, known therapeutic agents are unable to successfully treat various types of cancer. Cancer treatment is complicated by the relatively few compounds or agents that kill cancer cells and can be safely administered to cancer patients.

### SUMMARY OF THE INVENTION

According to an aspect of the invention, methods for reducing cell viability in a population of malignant cells are provided. The methods including: contacting a population of cells comprising malignant cells with two or more of Thiostrepton (TS), gentian violet (GV), and TLK-199, under conditions and for a period of time sufficient for substantial reduction in viability of at least a portion the malignant cells. In some embodiments, the method also includes providing a population of malignant cells. In some embodiments, the TLK-199 potentiates an activity of one or more of TS and GV. In some embodiments, the potentiated activity comprises reducing the malignant cell viability. In some embodiments, the contacting comprises delivering a composition comprising two or more of TS, GV, and TLK-199 to the population of cells. In some embodiments, the contacting comprises delivering a composition comprising TS and TLK-199 to the population of cells. In some embodiments, the contacting comprises delivering TS and TLK-199 to the population of cells with the TS and TLK-199 in different compositions. In some embodiments the contacting comprises delivering two or more compositions comprising two or more of TS, GV, and TLK-199 to the population of cells. In some embodiments, two or more of the TS, GV, and TLK-199 are delivered as part of a composition, wherein the composition optionally further comprises one or more pharmaceutically acceptable carriers. In some embodiments, the population of cells is in a subject, and the contacting comprises administering the two or more of TS, GV, and TLK-199 to the subject. In some embodiments, the administering comprises systemic administration of two or more of the TS, GV, and TLK-199 to the subject. In some embodiments, the systemic administration comprises enteral administration or parenteral administration. In some embodiments, the quantity of TS administered in a 24 hour period ranges from 10 ng to 1 g. In some embodiments, the quantity of GV administered in a 24 hour period ranges from 10 ng to 1 g. In some embodiments, the quantity of TLK-199 administered in a 24 hour period ranges from 10 ng to 1 g. In some embodiments, the contacting comprises contacting with one or more compositions comprising one or more of TS, GV, and TLK-199. In some embodiments, the molar concentration of the TS in the composition is from 10 nM to 500 μM. In some embodiments, the molar concentration of the GV in the composition is from 10 nM to 500 μM. In some embodiments, the molar concentration of the TLK-199 in the composition is from 10 nM to 500 μM. In some embodiments, the composition also includes one or more of a pharmaceutically acceptable carrier and excipient. In some embodiments, the population of cells comprises malignant mesothelioma cells. In some embodiments, the population of cells comprises ovarian cancer cells. In some embodiments, the subject is a mammal, and optionally is a human. In some embodiments, the malignant cells are located in the subject in one or more of: the pleura of the lungs, the peritoneum, the pericardium, and the tunica vaginalis. In some embodiments, the administration comprises a primary treatment provided to the subject. In some embodiments, the subject additionally is administered one or more of a surgical regimen, a radio-therapy regimen, and a chemotherapy regimen. In some embodiments, the malignant cell is a cancer cell.

According to an aspect of the invention, compositions that include a Thiostrepton (TS) compound and a TLK-199 compound or functional TLK-199 derivative are provided. In some embodiments a functional derivative of TLK-199 is TLK-117. In some embodiments, the composition also includes a gentian violet (GV) compound. In some embodiments, the molar concentration of the TS compound in the composition is from 10 nM to 500 μM. In some embodiments, the molar concentration of the GV compound in the composition is from 10 nM to 500 μM. In some embodiments, the molar concentration of the TLK-199 compound in the composition is from 10 nM to 500 μM. In some embodiments, the composition also includes one or more of a pharmaceutically acceptable carrier and excipient.

According to an aspect of the invention, compositions that include Thiostrepton (TS) or a functional derivative thereof and TLK-199 or a functional derivative thereof are provided. In some embodiments a functional derivative of TLK-199 is TLK-117. In some embodiments, the composition also includes gentian violet (GV) or a functional derivative thereof. In some embodiments, the molar concentration of the TS or TS functional derivative in the composition is from 10 nM to 500 μM. In some embodiments, the molar concentration of the GV or the GV functional derivative in the composition is from 10 nM to 500 μM. In some embodiments, the molar concentration of the TLK-199 or the TLK-199 functional derivative in the composition is from 10 nM to 500 μM. In some embodiments, the composition also includes one or more of a pharmaceutically acceptable carrier and excipient.

### BRIEF DESCRIPTION OF THE DRAWINGS

FIG. 1 provides a graph showing IC50 of malignant mesothelioma (MM) cells treated with thiostrepton (TS) alone or in combination with indicated concentrations of TLK-199.

FIG. 2A-G provides graphs of IC50 results. FIG. 2A shows results for TS and for TS/GV combined in a 10:1 ratio. FIG. 2B shows results for TS and for TS+10 μM TLK-199. FIG. 2C shows results for TS and for TS+20 μM TLK-199. FIG. 2Dd shows results for TS and for TS+30 μM TLK-199. FIG. 2E shows results for TS/GV (10:1) and for TS/GV+10 μM TLK-199. FIG. 2F shows results for TS/GV (10:1) and for TS/GV+20 μM. FIG. 2G shows results for TS/GV (10:1) and for TS/GV+30 μM.

FIG. 3A-E provides graphs and a table showing cell viability results. FIG. 3A shows results of testing in mesothelial (LP9) cells. FIG. 3B shows results of testing in malignant mesothelioma cells (HM cell line). FIG. 3C shows results of testing in malignant mesothelioma cells (MI cell line). FIG. 3D shows results of testing in malignant mesothelioma cells (PET cell line). FIG. 3 is a table showing results when cells of the >P9, HM, MI, and PET cell lines are treated with various compositions and amounts listed across the top of the table.

FIG. 4A-B provides a photomicrographic image and a graph showing results of certain studies testing effects of combining thiostrepton (TS) with TLK-199 on covalent adduction of peroxiredoxin 3 (PRX3). FIG. 4A shows representative western blot results of PRX3 protein. In the presence of TS PRX3 runs as a monomer (PRX3) and a covalently adducted non-reducible dimer (PRX3-TS-PRX3). FIG. 4B shows that the ratio of PRX3 dimer (PRX3-TS-PRX3) to PRX3 monomer (PRX3) is a quantitative assessment of TS activity on PRX3 protein. In the presence of TLK-199, TS activity is significantly increased compared to TS alone at equal concentrations (n=2, *p<0.05, **p<0.01, One-Way ANOVA with a Tukey's multiple comparisons test).

### DETAILED DESCRIPTION

Due to the complex and varied nature of cancers it has remained difficult to understand the diverse physiological mechanisms that may play a role in the development of cancer and/or its treatment. It has now been determined that contacting cancer cells with two or more of Thiostrepton (TS), gentian violet (GV), and TLK-199 results in a potentiated anti-cancer therapeutic effect as compared to the effect of contacting a cancer cell with TS or TS and GV in the absence of TLK-199. The effect of contacting a cancer cell or plurality of cancer cells with two or three of the agents: TS and GV and TLK-199 results in a synergistic effect of the cancer cell killing effects compared to contacting the cancer cells with the agents individually and also compared to contacting the cancer cells with the combination of TS and GV in the absence of the TLK-199. Such a synergistic effect of administering two or three of TS, GV, and TLK-199 to a cancer cell and/or a subject with cancer can result in a synergistic treatment effect in which more cancer cell death results from the treatment than occurs when TS is administered alone, GV is administered alone, TS and GV are both administered to the cancer cell and/or subject with cancer. Certain aspects of the invention include contacting one or more cancer cells with two or three of the agents GV, TS, and TLK-199 to treat cancer in a cell, tissue, or subject.

Thiostrepton is an antibiotic compound that is also known by other names including, but not limited to: Bryamycin, Thiactin, alaninamide, HR4S203Y18, etc. Gentian violet (GV), also known as crystal violet and methyl violet, is a triphenylmethane dye with anti-bacterial, anti-fungal, anti-helminithic, anti-trypanosomal, anti-angiogenic and anti-tumor properties. TLK-199, chemical name: (S)-ethyl 2-amino-5-(((R)-3-(benzylthio)-1-(((S)-2-ethoxy-2-oxo-1-phenylethyl)amino)-1-oxopropan-2-yl)amino)-5-oxopentanoate, is also known as Ezatiostat hydrochloride. TLK-199 has been used as a treatment for bone marrow disorders, such as myelodysplastic syndrome (MDS). In some embodiments of the invention a functional derivative of a TS, TLK-199, or GV compound may be used. A functional derivative is a derivative of the parent compound that possesses at least a portion of the function of the parent when used in methods as described herein. In some embodiments a functional derivative of TLK-199 is TLK-117 [see Townsend, D. M. & Tew, K, D., Oncogene 22:7369-7375(2003), the entire content of which is incorporated herein by reference.] Other art-known functional derivatives may be suitable for use in compositions and methods of the invention.

The invention, in part, pertains to methods of contacting cancer cells with two or three of the agents: TS, GV, and TLK-199 to treat cancer in cells, tissues, and subjects. In certain aspects of the invention comprises a composition comprising one or more of TS, GV, and TLK-199, (also referred to herein as “agents”). In certain aspects of the invention a composition that in includes one, two, or three of TS, GV, and TLK-199 also comprises one or more targeting agents or delivery agents. In certain embodiments of the invention treatment with a composition comprising one, two, or three of the agents: TS, GV, and TLK-199 comprises contacting one or more cancer cells with the agent or agents, wherein the contact results in the death of one or more of the contacted cancer cells. In certain aspects of an invention, a combination of TS and TLK-199 is administered to a cancer cell to treat the cancer. In certain embodiments of the invention, a combination of TS, GV, and TLK-199 is administered to a cancer cell to treat the cancer.

Molecules, compounds, compositions, and methods of the invention may be used to treat a subject who has or is considered to be at risk of having a cancer. Thus, methods and compounds of the invention are useful to treat cancers in cells and in subjects. Cells that may be treated using a combination comprising two or three of the agents TS, GV, and TLK-199 include but are not limited to cancer cells. A cancer cell that is treated using a method, compound, or composition of the invention may be in vitro or in vivo. An in vivo cell may be in a subject. As used herein the term “one or more” when used in reference to a cell means a single cell or a plurality of cells. A plurality of cells includes at least 2, 3, 4, 5, 6, 7, 8, 9, 10, 20, 50, 100, 500, 1,000, 5,000, 10,000, 50,000, 100,000, 500,000, 1,000,000, 5,000,000, 10,000,000, or more cells, including all integers in between. In some aspects of the invention a cell or plurality of cells is in cell culture or is in a subject. A plurality of cells may be a homogeneous or heterogeneous set of cells. As used herein, a homogeneous plurality of cells means a plurality of cells in which one or more cell characteristics of interest are the same for all of the cells in the plurality. It will be understood that a homogeneous plurality of cells may be considered to be homogeneous on the basis that each of the cells has the one or more characteristics of interest that are the same, for example are cancer cells, but the cells need not be entirely identical and features or characteristics, other than the characteristics of interest may differ in different cells of the plurality. In certain aspects of the invention, a plurality of cells is in a subject.

As used herein, a subject shall mean a vertebrate animal including but not limited to a human, mouse, rat, guinea pig, rabbit, cow, dog, cat, horse, goat, and non-human primate, e.g., monkey. In certain aspects of the invention, a subject may be a domesticated animal, a wild animal, or an agricultural animal. Thus, the invention can be used to treat diseases or conditions in human and non-human subjects. For instance, methods and compositions of the invention can be used in veterinary applications as well as in human prevention and treatment regimens. In some embodiments of the invention, the subject is a human. In some embodiments of the invention, a subject has one or more cancers.

A combination of TS, GV, and TLK-199 may be administered to a cell, tissue, and/or subject to treat one or more different types of cancer, including but not limited: a malignant mesothelioma, an ovarian cancer, a carcinoma, a sarcoma, a leukemia, a lymphoma, a myeloma, a glioma, breast cancer, an epithelial cancer, uterine cancer, vaginal cancer, prostate cancer, testicular cancer, penile cancer, colon cancer, rectal cancer, cervical cancer, throat cancer, oral cancer, pancreatic cancer, kidney cancer, liver cancer, lung cancer, melanoma, basal cell carcinoma, squamous cell carcinoma, head and neck cancer, stomach cancer, bone cancer, connective tissue cancer, bladder cancer, ocular cancer, nasal cancer, adipose cancer, thyroid cancer, non-Hodgkin lymphoma, small intestine cancer, Wilms tumor, gastrointestinal cancer, central nervous system (CNS) cancer, peripheral nervous system (PNS) cancer, esophageal cancer, Karposi sarcoma, gallbladder cancer, Hodgkin lymphoma, multiple myeloma, osteoscarcoma, neuroblastoma, rhabdomyoscarcoma, sinus cancer, retinoblastoma, and salivary cancer.

A combination of two or three of the agents TS, GV, and TLK-199 may be utilized and administered to treat numerous different types of cells, including but not limited to: a pleural cell, a dermal cell, a breast tissue cell, a muscle cell, a circulatory cell, a connective tissue cell, a bone cell, an exocrine cell, an endocrine cell, an organ cell, a mesenchyme cell, a connective tissue cell, an epithelial cell, an endothelial cell, a neuronal cell, a glial cell, a glandular cell, a stromal cell, a renal cell, a thyroid cell, a stem cell, a hematopoietic cell, a lymphoid cell, a myeloid cell, an erythroid cell, a cardiomyocyte, an hepatocyte, an astrocyte, an oligodendrocyte, and an adipocyte. In certain aspects of the invention a cell treated by a method comprising a contacting a cell with a combination of two or three of TS, GV, and TLK-199 compound of the invention may be a cancer cell, and the cancer may be any of the aforementioned cancers or other art-known cancers or neoplasms. In some aspects of the invention the cancer is a malignant mesothelioma and in certain embodiments of the invention the cancer is an ovarian cancer.

In certain aspects of the invention a plurality of cancer cells comprises a tumor that includes cancer cells in spatial proximity to each other. In some aspects of the invention a plurality of cells may not be in spatial proximity to each other, for example they may be in a subject but may be spatially separated in two or more different spatial regions, tissues, and/or organs of the subject. In some aspects of the invention, a plurality of cancer cells comprises one or more of a primary cancer and a secondary cancer (for example a metastasis); one or more primary cancers; and/or one or more secondary cancers. As used herein a primary cancer is used in reference to the initial, originating cancer and a secondary cancer comprises cancer cells that have broken away from a primary cancer and traveled to and are present in a different tissue or organ in the subject. A cancer cell from a secondary cancer may be the same cancer type as the originating primary cancer. In certain embodiments, a subject may have more than one cancer type. In certain embodiments of the invention a cancer cell is a metastatic cancer cell.

Non-limiting examples of subjects to which methods and compositions of the present invention can be applied are subjects who have been diagnosed with, are suspected of having, or are at risk of having one or more cancers. Methods of the invention may be applied to a subject who, at the time of treatment using a method and combination of two or three of the agents: TS, GV, and TLK-199 has been diagnosed with a cancer and is (1) undergoing treatment for one or more cancers, (2) has undergone treatment for one or more cancers, and/or (3) will be administered a treatment for one or more cancers.

In some aspects of the invention, a subject is at risk of having or developing one or more cancers. A subject at risk of developing a cancer has an increased probability of developing the cancer, compared to a control risk of developing the cancer or having a metastatic cancer. In some embodiments of the invention, a level of risk may be statistically significant compared to a control level of risk. A subject at risk may include, for instance, a subject having a genetic abnormality, the presence of which has been demonstrated to have a correlative relation to a higher likelihood of developing a cancer; a subject who has undergone a treatment for a primary cancer (for example, but not intended to be limiting, removal of a primary tumor) but who considered to be at risk for a secondary cancer and/or metastasis and/or another type of cancer; a subject undergoing cancer treatment other than a cancer treatment of the invention; a subject having a family and/or personal medical history of one or more cancers; a subject exposed to agents such as asbestos, chemical toxins, or activities; and/or a subject who has previously been treated for the cancer and is in apparent remission.

The effectiveness of a treatment comprising administering two or three of the agents: TS, GV, and TLK-199 can be determined and compared to control values. A control value may be a predetermined value, which can take a variety of forms. It can be a single cut-off value, such as a median or mean. It can be established based upon comparative groups, such as in groups of cells or individuals that have a cancer and are not treated with the combination of two or three of TS, GV, and TLK-199. Another comparative group may be a group of subjects with a history of a cancer and a group of subjects without such a history. The predetermined value will depend upon the particular population selected. Accordingly, the predetermined value selected may take into account the category in which an individual or cell falls. Appropriate ranges and categories can be selected with no more than routine experimentation by those of ordinary skill in the art. As used herein, effectiveness means significantly improved as compared to a control having a cancer but not treated with the combination of two or three of TS, GV, and TLK-199. Effectiveness may mean a level of cell death of cancer cells resulting from the administration of a combination of two or three of TS, GV, and TLK-199. A level of cell death in a subject treated with a combination two or three of TS, GV, and TLK-199 using a method of the invention may be compared to a control subject not treated with that combination and the effectiveness may be seen as an increase in number of cells that die, that is more than zero, or more than 1, 2, 3, 4, 5, 6, 7, 8, 9, 10, or more times more than the number of cells killed in the control that is not treated with a combination of the invention.

It will be understood that controls according to the invention may be, in addition to predetermined values, samples of materials tested in parallel with the experimental materials. Examples include samples from control populations or control samples generated through manufacture to be tested in parallel with the experimental samples; and also a control may be a sample from a subject prior to, during, or after a cancer treatment, including but not limited to a treatment of the invention.

### Treatment

As used herein, the terms “treat”, “treated”, or “treating” when used with respect to a cancer may refer to a prophylactic treatment that decreases the likelihood that a subject developing the cancer, and also may refer to a treatment after the subject has developed the cancer in order to eliminate or ameliorate the cancer, prevent the cancer from becoming more advanced (e.g., metastasizing, spreading, enlarging, etc.), and/or slow the progression of the cancer compared to progression in the absence of the therapy.

In certain embodiments of the invention, contacting a cancer cell with a combination of two or three of TS, GV, and TLK-199 increases the likelihood of death of the cancer cell, versus a cell not contacted with the combination.

### Administration Strategies

The each of the two or three TS, GV, and TLK-199 may be administered singly or in combination with one or both of the other compounds. The two or three of TS, GV, and TLK-199 need not be administered in a single pharmaceutical composition or using a single administration means. In some aspects of the invention two or three of the TS, GV, and TLK-199 agents may be in separate compositions or may be in one composition. In some aspects of the invention, when the two or three TS, GV, and TLK-199 agents are in two or three separate compositions, the two or three compositions may be administered at the same times, or may be administered sequentially, using a regimen in which their administration overlaps in part or in full. It will be understood that in some aspects of the invention the two or three of TS, GV, and TLK-199 are administered at the same time and in certain embodiments of the invention, two or three of TS, GV, and TLK-199 are in different compositions when administered. In a non-limiting example, in some embodiments of the invention, TS and TLK-199 are in one pharmaceutical composition and are administered to a subject together, and the GV is in a different pharmaceutical composition that is delivered either as the same time as, or at a different time than the TS and TLK-199 are administered to the subject. The synergistic effect of administering TS, GV, and TLK-199 need not require their administration in a single composition. In another non-limiting example, TS and TLK-199 are provided in the same pharmaceutical composition and are administered together to a subject and GV is not administered to the subject as part of the treatment.

In some embodiments, two or three of TS, GV, and TLK-199 administered to a subject with cancer who is also administered one or more other therapeutic agents and treatments. Thus, for example, administration of two or three of TS, GV, and TLK-199 may be undertaken in conjunction with chemotherapeutic agent and may enhance the cancer cell killing efficacy of the chemotherapeutic agent. A non-limiting example of a chemotherapeutic agent is: a taxane, an anthracyclines, a platinum-based drug, an anti-metabolite, a base analog, a nucleoside analogue, a nucleotide analogue, an antifolate, methotrexate, an alkaloid, vincristine, vinblastine, irinotecan, etoposide, velcade, a tyrosine kinase inhibitor, a serine/threonine kinase inhibitor, bleomycin, cyclophosphamide, cytoxan, everolimus, and metformin. In certain aspects of the invention, the two or three TS, GV, and TLK-199 agents that are included methods and compositions of the invention are in conjunction with other molecules such as targeting agents and labeling agents in treatment methods of the invention. A targeting agent or labeling agent may be attached to a TS, GV, or TLK-199 compound of the invention using art-known means such as, but not limited to via a covalent bond. In some aspects of the invention a targeting or labeling agent may be incorporated into TS, GV, and/or TLK-199 when it is produced or may be added post-production using routine chemical manufacturing methods.

Targeting agents useful in some aspects of the invention are targeting agents that direct or assist in directing one or more of TS, GV, and TLK-199 to a specific cell type to be treated such as a dermal cell, ovary tissue cell, a plural cell, a breast tissue cell, a muscle cell, a stem cell, a circulatory cell, a connective tissue cell, a bone cell, an exocrine cell, an endocrine cell, an organ cell, a mesenchyme cell, a connective tissue cell, an epithelial cell, an endothelial cell, a neuronal cell, a glial cell, a glandular cell, a stromal cell, a renal cell, a thyroid cell, a stem cell, a hematopoietic cell, a lymphoid cell, a myeloid cell, an erythroid cell, a cardiomyocyte, an hepatocyte, an astrocyte, an oligodendrocyte, an oocyte, or an adipocyte. Certain targeting agents useful in some aspects of the invention may be agents that direct or assist in directing one, two, or three of a TS, a GV, and a TLK-199 compound to an organelle such as a mitochondrion.

A targeting agent of choice will depend upon the nature of the cancer. In some instances it may be desirable to target the combination of agents to one or more pleural regions, lung cells, dermal cells, breast tissue cells, muscle cells, circulatory cells, connective tissue cells, stem cells, bone cells, exocrine cells, endocrine cells, organ cell, mesenchyme cells, connective tissue cells, epithelial cells, endothelial cells, neuronal cells, glial cells, glandular cells, stromal cells, renal cells, thyroid cells, stem cells, hematopoietic cells, lymphoid cells, myeloid cells, erythroid cells, cardiomyocytes, hepatocytes, astrocytes, oligodendrocytes, oocytes, and adipocytes. Those of ordinary skill in the art will be aware of and will be able to select and use suitable targeting agents in embodiments of the invention using routine methods. A non-limiting example of a targeting agent useful in certain embodiments of the invention is a cell-penetrating peptide, a cell internalization agent, sequence, a small molecule, a polynucleotide, a liposome, a PEGylated liposome, an aquasome, a biodegradable polymer, a nanoparticle, an oligonucleotide, and other targeting polypeptides.

The invention in some aspects includes a targeting agent of one or more of TS, GV, and TLK-199 to mitochondria. A non-limiting example of a mitochondrial targeting agent is a Gramicidin S based mitochondrial targeting agent, a mitochondria-targeting peptide; a nanoparticle that traffics to mitochondria, and a liposome-based delivery systems for mitochondria, an agent utilizing the carnitine-acylcarnitine translocase system, cytochromes, and malate dehydrogenase. Additional mitochondrial targeting agents that may be used in methods of the invention to target one, two, or three of TS, GV, and TLK-199 to the mitochondria are MitoQ and Triphenylphosphonium (TPP). Additional examples of targeting signals that may be used in some embodiments of the invention are set forth in Diekert, K., et al., PNAS (1999) vol 96, No. 21, 11752-11757; Murphy, M. Biochimica et Biophysica Acta 1777 (2008) 1028-1031; Addya, S., et al., J. Cell Biology, (1997) Vol. 139, No. 3, 589-599; Del Gaizo, V., et al., (2003) Mol. Gen. and Metabol., Vol. 80, 170-180, which are incorporated herein by reference.

Labeling agents may be used in certain embodiments of methods and compounds of the invention to determine the location of one, two, or three of TS, GV, and TLK-199 compounds in cells and tissues and also, may be used to assess the cell, tissue, or organelle location of treatment compounds that have been administered. Procedures for attaching and utilizing labeling agents such as enzymatic labels, dyes, radiolabels, fluorescent labels, etc. are well known in the art.

Compositions, compounds, and methods of the invention may be enhanced by utilization in combination with other procedures for treating a cancer. In some instances a treatment procedure may involve administration of another therapeutic agent or treatment such a medicament and/or surgery, radiation therapy, etc. Thus, in some embodiments of the invention, administration of two or three of TS, GV, and TLK-199 is performed at one of more of: prior to, coincident with, or after administration of another therapy for treating the cancer. Treatment methods of the invention that include administration of two or three of a TS, a GV, and a TLK-199 compound can be used at any stages of a cancer including in a pre-cancer, dysplasia, tumor, metastasis, remission, relapse, etc. Methods of the invention may also be used for subjects who have previously been treated with one or more other anti-cancer medicament, chemotherapeutic, surgery, or radiation methods that were not successful, were minimally successful, and/or are no longer successful at slowing or stopping progression of the cancer in the subject.

### Effective Amounts

Two or three of a TS, a GV, and a TLK-199 compound of the invention are administered to a cell or subject in an effective amount for treating a cancer. An “effective amount for treating a cancer” is an amount necessary or sufficient to realize a desired biologic effect. For example, an effective amount of two or three of TS, a GV, and a TLK-199 compound could be that amount necessary to do one or more of (i) slowing or halting progression of the cancer; (ii) killing a plurality of cancer cells, and (iii) reversing one or more symptoms of the cancer. According to some aspects of the invention, an effective amount is that amount of each of two or three TS, GV, and TLK-199 compounds as a combination alone or in further combination with another medicament or treatment, which when combined or co-administered or administered alone, results in a desired therapeutic response in the cancer, either in the prevention or the treatment of the cancer. In some aspects of the invention, a desired biological effect may be one or more of: death of a plurality of cancer cells; the amelioration and or absolute elimination of symptoms resulting from the cancer; a partial or complete abrogation of the cancer, as evidenced for example, by a diagnostic test that indicates the subject is free of the cancer, or that one or more of the presence, level, or tumor size, and severity of the cancer is reduced.

Typically an effective amount of two or three of TS, GV, and TLK-199 compounds will be determined in clinical trials, establishing an effective dose for a test population versus a control population in a blind study. In some embodiments, an effective amount will be that results in a desired response, e.g., an amount that diminishes a cancer; increases chemo-sensitivity of one or more cancer cells, maintains a cancer in remission in cells and/or a subject with the cancer. Thus, an effective amount to treat a cancer may be the amount that when administered increases cell death of a plurality of cancer cells in the subject at an amount that is above the amount that would occur in the subject or tissue without the administration of the two or three TS, GV, and TLK-199 compounds in a method of the invention. In the case of treating a cancer a desired response to a treatment of the invention may be reducing or eliminating one or more symptoms or physiological characteristics of the cancer in a cell, tissue, and/or subject. The reduction or elimination may be temporary or may be permanent. The status of the cancer can be monitored using art-known methods. In some aspects of the invention, a desired response to treatment of a cancer may comprise delaying or preventing onset of the cancer, slowing, delaying, or stopping a cancer's progression, maintaining remission of a cancer, etc.

An effective amount of a combination of two or three of TS, GV, and TLK-199 in a treatment method of the invention may also be determined by assessing physiological effects of administration of the combination of the two or three compounds on a cell or subject, such as a an increase in cancer cell death, a decrease of the cancer, etc. following administration. As herein the term “administrating” when used in reference to treating one or more cancer cells means contacting the one or more cancer cells with a combination of two or three of TS, GV, and TLK-199. In certain embodiments of the invention, TS, GV, and TLK-199 compounds are part of a pharmaceutical composition. In certain embodiments of the invention, TS and TLK-199 compounds are part of a pharmaceutical composition. In certain embodiments of the invention, a TS compound is part of a pharmaceutical composition. In certain embodiments of the invention, two or three of TS, GV, and TLK-199 may be administered as part of one, two, or three different pharmaceutical compositions, wherein the manner of administration of each of the compositions is suitable to contact one or more cancer cells with the TS, GV, and/or TLK-199 compound. Some embodiments of pharmaceutical compositions of the invention include one, two, or three of a: TS, GV, and TLK-199 compound and also include a pharmaceutically acceptable carrier. It will be understood that if one or more of TS, GV, and TLK-199 are part of separate pharmaceutical compositions, each of the pharmaceutical compositions, can but need not include the same pharmaceutically acceptable carrier.

Assays suitable to determine efficacy of a combination of two or three of TS, GV, and TLK-199 will be known to those skilled in the art and can be employed for measuring the level of the response to a treatment and an amount and dosage of the combination of the two or three compounds administered to a subject. The amount of one, two, or three of TS, GV, and TLK-199 can be modified based, at least in part, on such measurements. Non-limiting examples of measurements of response to a cancer treatment of the invention include cancer diagnostic testing, staging, tumor measure, scans, etc. The amount of a treatment may be varied for example by one or more of: increasing or decreasing the amount of a pharmaceutical composition administered, changing the pharmaceutical composition administered, changing the route of administration, changing the dosage timing, changing administration of another therapeutic agent, a non-limiting example of which is a chemotherapeutic agent, and so on. The effective amount will vary with the particular cancer being treated, the age and physical condition of the subject being treated; the stage and severity of the cancer, the duration of the treatment, the nature of a prior, concurrent, or impending therapy (if any), the specific route of administration, and additional factors within the knowledge and expertise of the health practitioner. For example, though not intended to be limiting, an effective amount of TS may depend on the manner with which it is delivered, as well as whether or not GV or TLK-199 are also included in the pharmaceutical composition that includes the TS.

An effective amount of one or more of a combination of two or three of TS, GV, and TLK-199 for treatment of a cancer may vary depending upon the specific compounds included, the mode of delivery of the compounds, and whether the treatment is used alone or in combination with another therapeutic agent or compound. The effective amount for any particular application can also vary depending on such factors as the cancer being treated, the particular compound being administered, the size of the subject, or the severity of the metabolic disease or condition. A skilled artisan can empirically determine the effective amount of a particular compound of the invention without necessitating undue experimentation. Combined with the teachings provided herein, by choosing among the combinations of two or three of TS, GV, and TLK-199 and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat a cancer in a particular subject.

A pharmaceutical composition dosage and/or dosage of the two or three of TS, GV, and TLK-199 can be adjusted by an individual health care provider or veterinarian, particularly in the event of any complication. For use in a combination treatment method of the invention, a therapeutically effective amount of each of TS, GV, and TLK-199 are independently determined and typically vary from 0.01 mg/kg to about 1000 mg/kg, from about 0.1 mg/kg to about 200 mg/kg, or from about 0.2 mg/kg to about 20 mg/kg, in one or more dose administrations daily, for one or more days. The absolute amount will depend upon a variety of factors including a concurrent treatment, the number of doses and the individual subject parameters including age, physical condition, size and weight. These are factors well known to those of ordinary skill in the art and can be addressed with no more than routine experimentation. In some embodiments, a maximum dose can be used, that is, the highest safe dose according to sound medical judgment. Depending on the route of drug delivery (direct administration vs. systemic) the concentration of drug for clinical administration will vary.

Multiple doses of combinations of compounds of the invention are also contemplated. In some instances, TS and TLK-199 can be administered to a subject once, twice, three or more times, at least daily, every other day, weekly, every other week, monthly, etc. Doses may be administered once per day or more than once per day, for example, 2, 3, or more times in one 24 hour period.

Pharmaceutical compositions of the invention may be administered alone, in combination with each other, and/or in combination with other drug therapies, or other treatment regimens that are administered to subjects with a metabolic disease or condition. Pharmaceutical compositions used in the embodiments of the invention preferably are sterile and contain an effective amount of a combination of two or three of TS, GV, and TLK-199 to do one or more of: kill a contacted cancer cell and produce the desired therapeutic response in a unit of weight or volume suitable for administration to a subject.

The doses of a pharmaceutical composition and/or two or three of TS, GV, and TLK-199 treat a cancer that is administered to a subject can be chosen in accordance with different parameters, in particular in accordance with the mode of administration used and the state of the subject. Other factors may include the desired period of treatment. In the event that a response in a subject is insufficient at the initial doses applied, higher doses (or effectively higher doses by a different, more localized delivery route) may be employed to the extent that patient tolerance permits.

### Administration Methods

A variety of administration routes for a combination of two or three of TS, GV, and TLK-199 are available. The particular delivery mode selected will depend upon the particular condition being treated and the dosage required for therapeutic efficacy. Methods of this invention, generally speaking, may be practiced using any mode of administration that is medically acceptable, meaning any mode that produces effective levels of treatment without causing clinically unacceptable adverse effects. In some embodiments of the invention, a combination of two or three of TS, GV, and TLK-199 may be administered via an oral, enteral, mucosal, percutaneous, and/or parenteral route. The term “parenteral” includes subcutaneous, intravenous, intramuscular, intraperitoneal, and intrasternal injection, or infusion techniques. Other routes include but are not limited to nasal (e.g., via a gastro-nasal tube), dermal, vaginal, rectal, and sublingual. Delivery routes of the invention may include intrathecal, intraventricular, or intracranial. In some embodiments of the invention, one or more of TS, GV, and TLK-199 in a combination treatment of the invention may be placed within a slow release matrix and administered by placement of the matrix in the subject. In some aspects of the invention, one or more of TS, GV, and TLK-199 may be administered to a cell and/or subject using nanoparticles coated with a delivery agent that targets a specific cell or organelle, a non-limiting example of which is a mitochondrion.

One or more of TS, GV, and TLK-199 may be administered in formulations, which may be administered in pharmaceutically acceptable solutions, which may routinely contain pharmaceutically acceptable concentrations of salt, buffering agents, preservatives, compatible carriers, adjuvants, and optionally other therapeutic ingredients. According to methods of the invention, one or more of the TS, GV, and TLK-199 compounds may be administered in one or more pharmaceutical compositions. In general, a pharmaceutical composition comprises the compound of the invention and a pharmaceutically-acceptable carrier. Pharmaceutically acceptable carriers are well known to the skilled artisan and may be independently selected and utilized using routine methods. As used herein, a pharmaceutically-acceptable carrier means a non-toxic material that does not interfere with the effectiveness of the biological activity of the active ingredients, e.g., the ability of combination of two or three of TS, GV, and TLK-199 to kill a cancer cell.

Pharmaceutically acceptable carriers may include diluents, fillers, salts, buffers, stabilizers, solubilizers and other materials that are well-known in the art. Exemplary pharmaceutically acceptable carriers are described in U.S. Pat. No. 5,211,657 and others are known by those skilled in the art. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

In some embodiments of the invention, one or more of a: TS, GV, and TLK-199 compound is administered directly to a tissue. In some embodiments, the tissue to which the compound is administered is a tissue in which cancer is present or is likely to be present or to arise. Direct tissue administration may be achieved by washing a tissue in a subject with a solution that includes a combination of two or three of TS, GV, and TLK-199 compounds. Administration using a washing means may also be referred to as: lavage and may include delivering one, two, or three of TS, GV, and TLK-199 compounds to a subject by flowing a solution that contains the one, two, or three compounds into a subject in a manner that the solution contacts cancer cells in the subject. The contact between a subject's tissues and the one, two, or three of the TS, GV, and TLK-199 compounds may be maintained for a period of time, such as for at least 0.1, 0.5, 1, 2, 3, 4, 5, 6, 7, 8, 9, 10, 15, 20, 25, 30, 35, 40, 45, 50, 55, 60, 65, 70, 75, 80, 85, 90, 120, 180, 240, 300, 360, 420, or more minutes, including each integer within the listed range of times. Contact with a combination of two or three of TS, GV, and TLK-199 may also be achieved using direct injection of one or more of TS, GV, and TLK-199, or other art-known means. In some embodiments of the invention one, two, or three of TS, GV, and TLK-199 are administered to a subject using an intra-plural wash. It will be understood that a combination treatment of the invention may include contacting cells in a subject with one of TS, GV, and TLK-199 using a washing means and contacting cells in the subject with another of TS, GV, and TLK-199 using a different means, non-limiting examples of which are: IV administration, slow release administration, etc. A combination of two or three of TS, GV, and TLK-199 may be administered once, or alternatively may be administered in a plurality of administrations. If administered once or multiple times, each of the two or three TS, GV, and TLK-199 compounds may be administered by the same or by different routes. For example, the first (or the first few) administrations may be made directly into the affected tissue while later administrations may be systemic.

In embodiments of the invention in which one, two, or three of TS, GV, and TLK-199 compounds are to be administered systemically, the compound(s) may be formulated for parenteral administration by injection, e.g., by bolus injection or continuous infusion. Formulations for injection may be presented in unit dosage form, e.g., in ampoules or in multi-dose containers, with or without an added preservative. The pharmaceutical compositions may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day may be used as needed to achieve appropriate systemic or local levels of one, two, or three of the TS, GV, and TLK-199 compounds.

In yet other embodiments, a delivery vehicle is a biocompatible microparticle or implant that is suitable for implantation into the mammalian recipient. Exemplary bioerodible implants that are useful in accordance with this method are described in PCT Publication No. WO 95/24929 (incorporated by reference herein), which describes a biocompatible, biodegradable polymeric matrix for containing a biological macromolecule. Such delivery means are well known in the art and can be used to achieve sustained release of a compound of the invention in a subject, and may be selected not to degrade, but rather, to release by diffusion over an extended period of time.

Both non-biodegradable and biodegradable polymeric matrices can be used to deliver one, two, or three TS, GV, and TLK-199 compounds to a cell and/or subject. In some embodiments, a matrix may be biodegradable. Matrix polymers may be natural or synthetic polymers. A polymer can be selected based on the period of time over which release is desired, generally in the order of a few hours to a year or longer. Typically, release over a period ranging from between a few hours and three to twelve months can be used. The polymer optionally is in the form of a hydrogel that can absorb up to about 90% of its weight in water and further, optionally is cross-linked with multivalent ions or other polymers.

In certain embodiments of the invention, one, two, or three TS, GV, and TLK-199 compounds may be delivered using the bioerodible implant by way of diffusion, or by degradation of the polymeric matrix. Exemplary synthetic polymers for such use are well known in the art. Biodegradable polymers and non-biodegradable polymers can be used for delivery of one, two, or three TS, GV, and TLK-199 compounds using art-known methods. Bioadhesive polymers such as bioerodible hydrogels (see H. S. Sawhney, C. P. Pathak and J. A. Hubell in Macromolecules, 1993, 26, 581-587, the teachings of which are incorporated herein) may also be used to deliver one, two, or three TS, GV, and TLK-199 compounds of the invention for treatment. Additional suitable delivery systems can include time-release, delayed release or sustained-release delivery systems. Such systems can avoid repeated administrations of the one, two, or three TS, GV, and TLK-199 compounds, increasing convenience to the subject and the physician. Many types of release delivery systems are available and known to those of ordinary skill in the art. (See for example: U.S. Pat. Nos. 5,075,109; 4,452,775; 4,675,189; 5,736,152; 3,854,480; 5,133,974; and 5,407,686 (the teaching of each of which is incorporated herein by reference). In addition, pump-based hardware delivery systems can be used, some of which are adapted for implantation.

Use of a long-term sustained release implant may be particularly suitable for prophylactic treatment of subjects and for subjects at risk of developing a recurrent cancer. Long-term release, as used herein, means that the implant is constructed and arranged to delivery therapeutic levels of the active ingredient for at least 30 days, 60 days, 90 days or longer. Long-term sustained release implants are well-known to those of ordinary skill in the art and include some of the release systems described above.

Therapeutic formulations of one, two, or three, TS, GV, and TLK-199 compounds of the invention may be prepared for storage by mixing the TS, GV, and/or TLK-199 compounds having the desired degree of purity with optional pharmaceutically acceptable carriers, excipients or stabilizers [Remington's Pharmaceutical Sciences 21^{st} edition, (2006)], in the form of lyophilized formulations or aqueous solutions. Acceptable carriers, excipients, or stabilizers are nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

Non-limiting examples of formulations for delivery of one, two, or three of TS, GV and TLK-199 in one embodiment of a method of the invention include but are not limited to one or more of: TS, GV, TLK-199, VitE-TPGS, DMSO, normal saline, Kolliphor-EL, dextrose, and sodium chloride.

### Efficacy Determination and Assays

Certain aspects of the invention include methods to assess the efficacy of a combination of two or three of TS, GV, and TLK-199 compounds in treatment of a cancer. Such methods may include comparing the effect on a cancer test cell contacted with one, two, or three of the TS, GV, and TLK-199 compound to the status of a substantially similar cancer control cell that is not contacted with the one, two, or three compounds. A change in one or more of desirable effects such as, but not limited to: increased likelihood of cancer cell death indicates effectiveness of combination of two or three of the TS, GV, and TLK-199 compounds for treatment of cancer. In some embodiments of the invention, assay methods may include obtaining a biological sample from a subject, contacting with two or three of the TS, GV, and TLK-199 compounds, optionally contacting the cell with a chemotherapeutic agent and assessing the cell's response (e.g., increased likelihood of cell death, etc.). The test cell's response may be compared to a control cancer cell. As used herein a biological sample may be an in vitro biological sample, or may a sample that is detected (e.g., obtained) in vivo. As used herein, a biological sample may be a cell sample, tissue sample, blood sample, bodily fluid sample, subcellular sample, etc. A biological sample may include cells, tissues, or organelles and may include cell types such as but not limited to: dermal cells, cells from a plural space, ovary tissue cells, breast tissue cells, muscle cells, circulatory cells, connective tissue cells, stem cells, bone cells, exocrine cells, endocrine cells, organ cell, mesenchyme cells, connective tissue cells, epithelial cells, endothelial cells, neuronal cells, glial cells, glandular cells, stromal cells, renal cells, thyroid cells, stem cells, hematopoietic cells, lymphoid cells, myeloid cells, erythroid cells, cardiomyocytes, hepatocytes, astrocytes, oligodendrocytes, oocytes, and adipocytes. In some embodiments of the invention, a biological sample may comprise one or more cancer cells.

Assays to assess a cancer may include but are not limited to (1) characterizing the efficacy of a combination of two or three of TS, GV, and TLK-199 compounds in treating a cancer in a subject; (2) evaluating a combination treatment comprising administering two or three of TS, GV, and TLK-199 compounds and administering one or more chemotherapeutic agents, radiation treatments, surgical treatments, and other therapeutic treatments, (3) selecting a treatment for a cancer based at least in part on the determined efficacy of the combination of two or three of the TS, GV, and TLK-199 compounds; and (4) administering a combination of two or three of TS, GV, and TLK-199 to a subject as at least a portion of a treatment of a cancer in the subject. Thus, subjects can be characterized, treatment regimens can be monitored, treatments can be selected and diseases status can be better understood using embodiments of methods of the present invention.

The invention, in some aspects, includes various assays to determine the efficacy of a combination of two or three of TS, GV, and TLK-199 compounds administered to a cancer cell and/or subject. Methods of the invention that are useful to determine efficacy of a combination treatment of the invention in cells, tissues, subjects, and samples (e.g., from subjects, in culture, etc.), include, but are not limited to: diagnostic assays to determine cancer cell death, etc. Assessments of efficacy of a combination of two or three of TS, GV, and TLK-199 compounds to treat a cancer can be done in vitro, for example in cell culture, cell samples, cell suspensions, etc. or can be done in vivo, for example in a living subject using art-known cancer diagnostic assessments and tracking methods.

As will be appreciated by those of ordinary skill in the art, the evaluation of a treatment also may be based upon an evaluation of the symptoms or clinical end-points of a cancer and such evaluations can be used in conjunction with methods of the invention to assess the status of a cancer and/or the efficacy of a treatment of the invention for a cancer.

The following examples are provided to illustrate specific instances of the practice of the present invention and are not intended to limit the scope of the invention. As will be apparent to one of ordinary skill in the art, the present invention will find application in a variety of compositions and methods.

### EXAMPLES

### Example 1

Studies were performed to test effects of combined thiostrepton (TS) and TLK-199 on malignant mesothelioma tumor cell viability.

### Methods:

Malignant mesothelioma (MM) cells were incubated with increasing concentrations of TS (0-10 μM) and TLK-199 (0-200 μM) for 48 hours and then assayed for cell number (viability). The inhibitory concentration required to kill 50% of the cells (IC50) was determined.

### Results:

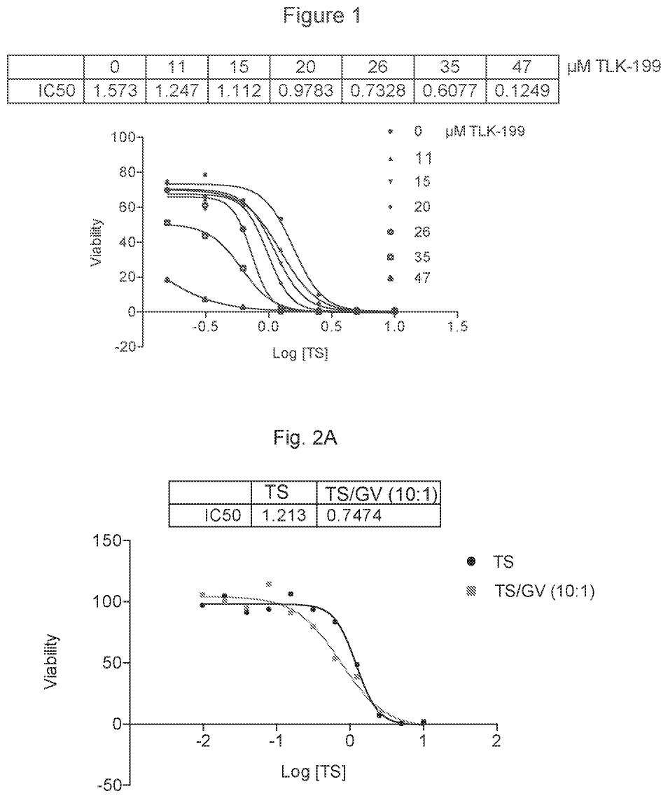

The inhibitory concentration required to kill 50% of cells (IC50) for TS was determined to be ˜1.5 μM. Addition of TLK-199 significantly reduced the IC50 of TS in a dose-dependent manner (FIG. 1). Results obtained in this study can be used in determining an amount of each of two or three of TS, GV, and TLK-199 to administer to a human subject for treatment of a cancer. A calculated IC50 for TS in malignant mesothelioma cell lines (7 human cell lines from both pleural and peritoneal biopsies) ranges from 0.9 μM to 2.4 μM and the calculated IC99 ranges from 6.8 μM to 36 μM. The IC50 for GV is 0.15 μM across all malignant mesothelioma cell lines. Combining TS with GV (10 parts TS: 1 part GV) reduced the IC50 of TS to 0.74 μM. The IC50 for TLK-199 in mesothelioma tumor cell lines is 45 μM. Combination of TS with increasing concentrations on TLK-199 induced synergistic cytotoxicity (See FIG. 1). Combining 30 μM TLK-199 with TS/GV (10 parts TS: 1 part GV) reduced the IC50 of TS/GV from 0.74 μM to 0.19 μM.

### Example 2

Studies were performed to test the effects on cell viability of mesothelioma (HM cell line) cells in culture to thiostrepton (TS) or TS+gentian violet (GV) in combination with TLK-199.

### Methods:

HM cells were plated into 96 well plates and allowed to adhere overnight. The following day test compounds (TS and TS/GV) were serially diluted in duplicate wells. TS concentrations ranged from 10 nM to 10 μM for TS alone treatment while TS/GV was serially diluted in a 10:1 ratio (stating at 10 μM TS:1 μM GV). TLK-199 was added at fixed concentrations of 0, 10, 20 or 30 μM to wells containing serially diluted TS or TS/GV, maintaining a fixed concentration of TLK-199 with varying TS or TS/GV concentrations.

### Results:

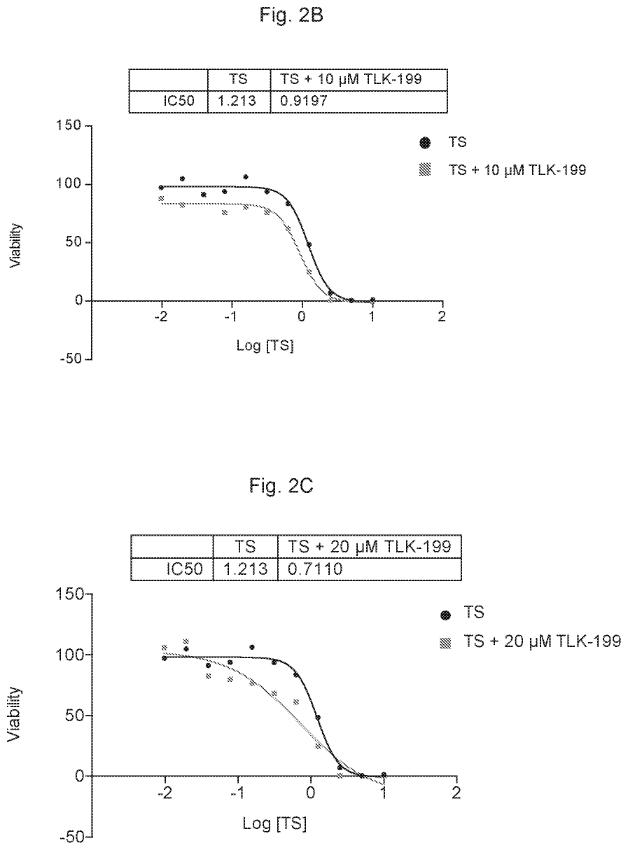

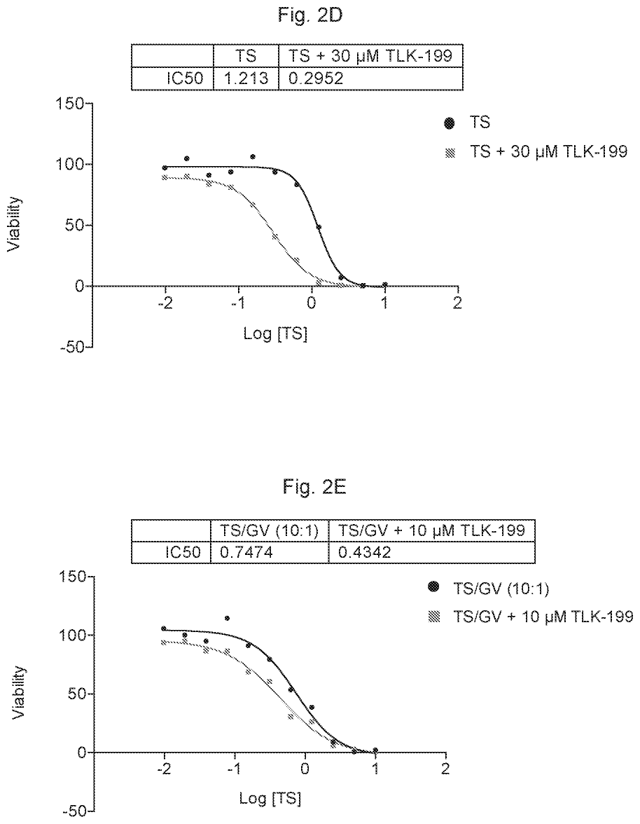

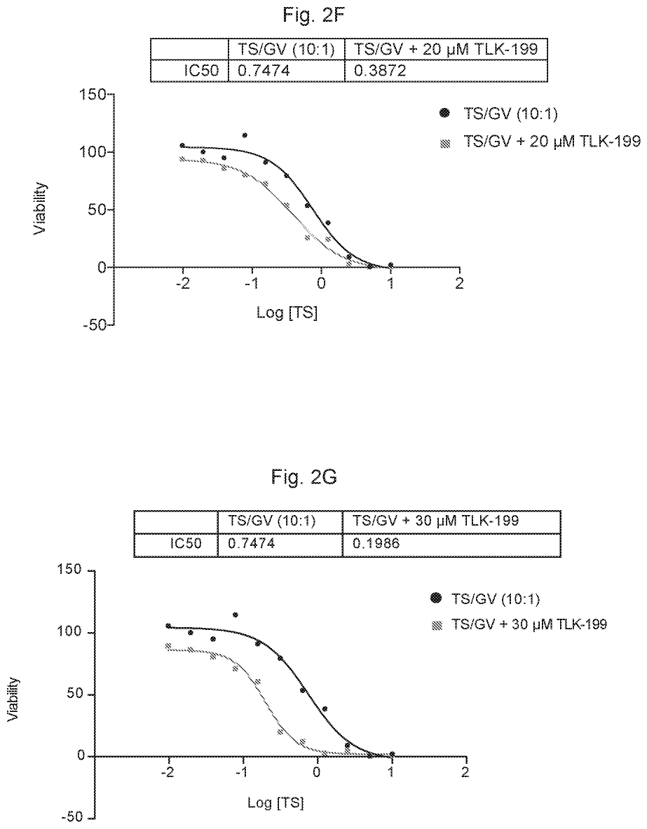

Cells incubated with TS/GV showed a reduction in IC50 (inhibitory concentration where 50% of the cells remain) compared to TS alone. The addition of TLK-199 to either TS or TS/GV potentiated the activity of TS alone and TS/GV, significantly reducing the IC50 compared to parent compounds. The combination of TS/GV/TLK-199 showed the largest effect on tumor cell viability. All calculated IC50s are tor [TS]. Results are shown in FIG. 2A-G. These studies were repeated and showed equivalent results.

### Example 3

Mouse experiments are performed that test the effects of combining TS and GV with TLK-199 on malignant mesothelioma (MM) tumor growth in vivo. Results indicate a reduction of viability of MM cells and a greater reduction in tumor growth than the effect of TS and GV without TLK-199.

### Example 4

Studies were performed to test the effects on cell viability of mesothelial (LP9) and malignant mesothelioma (HM , MI, PET cell line) cells in culture to thiostrepton (TS) or TS+gentian violet (GV) in combination with TLK-199. Methods:

Indicated cell lines were plated into 96 well plates and allowed to adhere overnight. The following day test compounds (TS/GV) were serially diluted in duplicate wells. TS/GV was serially diluted in a 10:1 ratio (stating at 10 μM TS:1 μM GV). TLK-199 was added at fixed concentrations of 0, 10, 20 or 30 μM to wells containing serially diluted TS or TS/GV, maintaining a fixed concentration of TLK-199 with varying TS or TS/GV concentrations.

### Results:

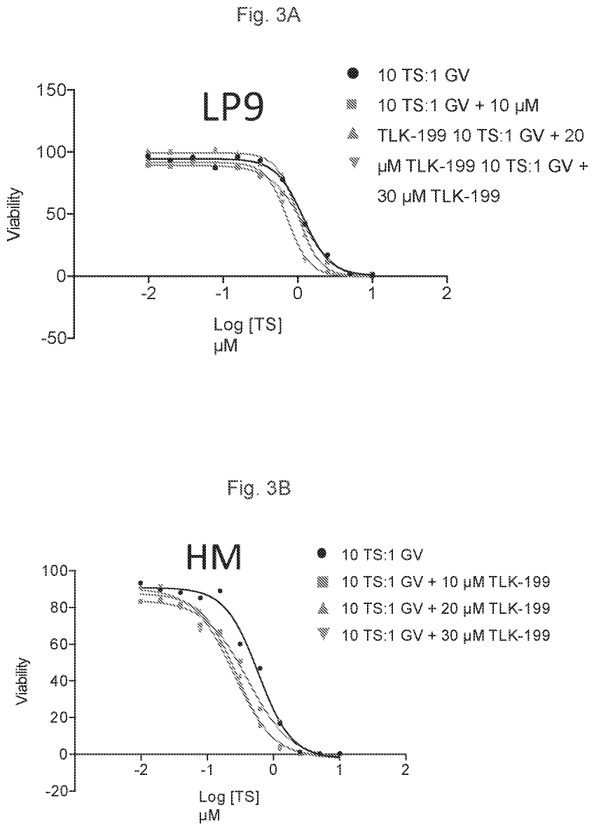

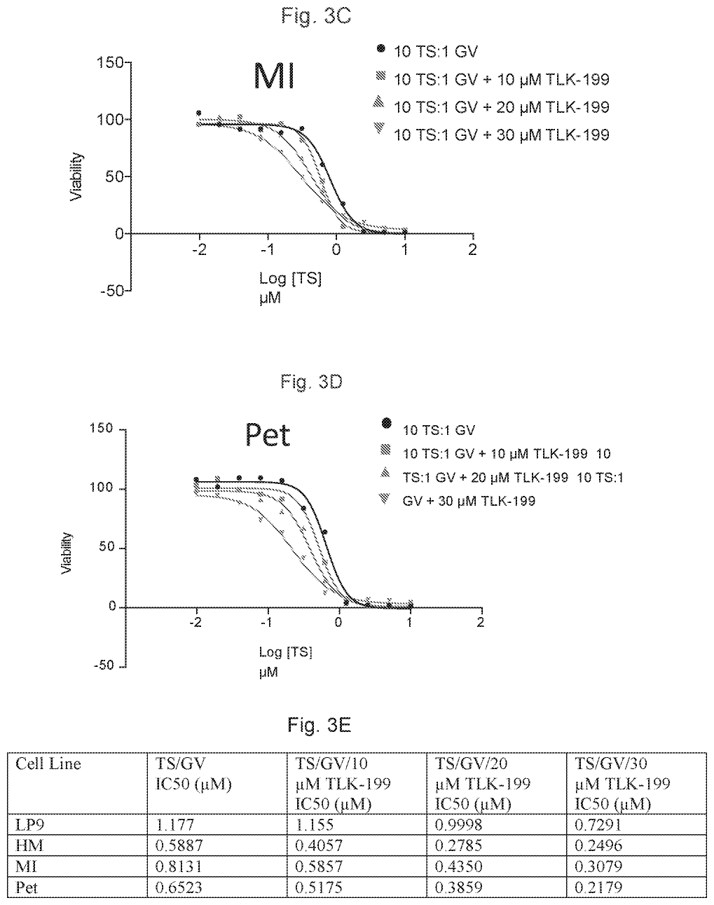

Cells incubated with TS/GV in combination with TLK-199 showed a reduction in IC50 (inhibitory concentration where 50% of the cells remain) compared to TS/GV alone. All calculated IC50s are for [TS]. Results are shown in FIG. 3A-E.

### Example 5

Different dilutions and formulations are prepared for administration of combination treatments that include two or more of TS, GV, and TLK-199. In a previous study, [Raza, A et al., Journal of Hematology 8 Oncology volume 2, doi:10.1186/1756-8722-2-20 Article number: 20 (2009)] subjects were administered 50, 100, 200, 400 or 600 mg/m² TLK-199 solution in a phase I dose escalation study. The solution was perfused IV for a duration of 60 minutes. Results from this showed improvement in clinical symptoms and reductions in transfusion requirements in patients with myelodysplatic syndrome. Improvement in bone marrow maturation and cellularity were also observed. Studies are performed to assess concentrations and formulations comprising TS.

The following tests are performed:

1. TS is diluted in 5% VitE-TPGS and 2% DMSO in 0.9% normal saline to a concentration of

2 mM. This solution is further diluted to the target dose concentration, which in some applications of methods of treatment is 10 μM for TS.

2. TS is diluted in 5% Kolliphor-EL and 2% DMSO in 0.9% normal saline to a concentration of 500 μM. This solution is further diluted to the target dose concentration, which in some applications of methods of treatment is 10 μM.

3. A TS/GV formulation is delivered to a human subject using an intra-pleural wash (time range is at least 5, 10, 15, 20, 25, 30, 40, 50, or 60 minutes in length and/or by systemic administration.

Formulations of TS such as those described in (1), (2), and (3) are administered with and without co-administrating TLK-199 to subjects with malignant mesothelioma. Results indicate the TS formulations reduce malignant mesothelioma (MM) tumor cell viability if administered to a subject and that the inclusion of TLK-199 enhances the tumor killing activity of the formulation in a synergistic manner.

### Example 6

Formulations of TS such as those described in Examples 1-5 are administered with and without co-administrating TLK-199 to subjects with at least one of an ovarian cancer, a breast cancer, an epithelial cancer, uterine cancer, a prostate cancer, a colon cancer, a lung cancer, and a gastrointestinal cancer.

Results indicate the administration of TS formulations reduce malignant cancer cell viability in a subject and the co-administration of TLK-199 enhances the can cell killing activity of the TS formulation in a synergistic manner.

### Example 7

Studies were performed to test the effects of combining thiostrepton (TS) with TLK-199 on covalent adduction of peroxiredoxin 3 (PRX3).

Methods

Malignant mesothelioma (MM) cells were incubated with 0, 2,and 4 μM Thiostrepton (TS) alone or in the presence of 30 μM TLK-199 for 24 hours. Cell lysates were collected, and proteins were separated by reducing SDS-Polyacrylamide Gel Electrophoresis.

Results

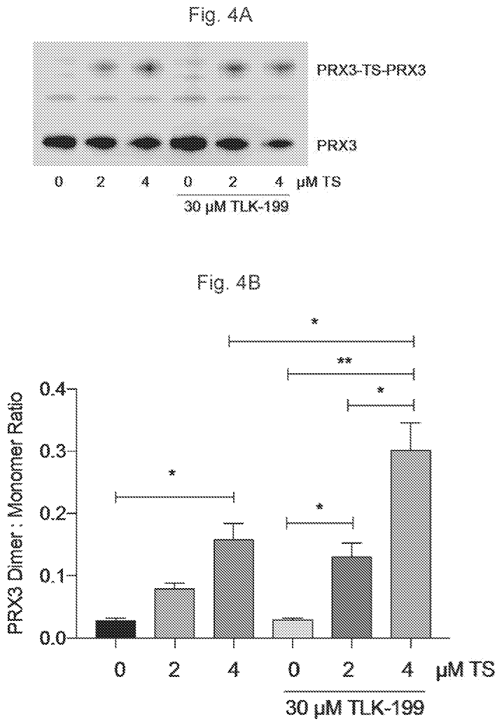

Under these conditions, PRX3 protein migrated as a single monomeric band (FIG. 4A, PRX3). In the presence of TS, in which PRX3 monomers were covalently crosslinked, a second band of equal molecular weight to a PRX3 dimer was present (FIG. 4A, PRX3-TS-PRX3). The ratio of PRX3-TS-PRX3 to PRX3 (PRX3 dimer:monomer ratio) was quantified in the presence of increasing concentrations of TS+/−TLK-199 (FIG. 4B). When TLK-199 was present, a statistically significant increase in the amount of PRX3 modified by TS was observed (FIG. 4B, 4 μM TS versus 4 μM TS+TLK-199). These data support previous viability assays that were performed in which MM cell viability in the presence of TS was accentuated when TLK-199 was included. These data support the synergistic activity of TS and TLK-199.

### Example 8

Subjects with malignant mesothelioma (MM) are administered (1) thiostrepton (TS) and TLK-199, (2) TS, gentian violet (GV), and TLK or (3) GV and TLK-199. The administration of (1), (2), and (3) results in reduced viability of MM cells in the subject. The TLK-190 results in a synergistic effect when it is included and increases the reduction in viability of MM cells in the subject.

### Equivalents

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated herein in their entirety herein by reference.

What is claimed is:

1. A method for reducing cell viability in a population of malignant cells, the method comprising: contacting a population of cells comprising malignant cells with two or more of Thiostrepton (TS), gentian violet (GV), and TLK-199, under conditions and for a period of time sufficient for substantial reduction in viability of at least a portion the malignant cells.

2. The method of claim 1, further comprising providing a population of malignant cells, optionally wherein the population of cells comprises malignant mesothelioma cells; and optionally wherein the population of cells comprises ovarian cancer cells.

3. The method of claim 1, wherein the TLK-199 potentiates an activity of one or more of TS and GV, optionally wherein the potentiated activity comprises reducing the malignant cell viability.

4. (canceled)

5. The method of claim 1, wherein the contacting comprises delivering a composition comprising two or more of TS, GV, and TLK-199 to the population of cells.

6. The method of claim 1, wherein two or more of the TS, GV, and TLK-199 are delivered as part of a composition, wherein the composition optionally further comprises one or more pharmaceutically acceptable carriers.

7. The method of claim 1, wherein the population of cells is in a subject, and the contacting comprises administering the two or more of TS, GV, and TLK-199 to the subject, optionally the administering comprises systemic administration of two or more of the TS, GV, and TLK-199 to the subject, and optionally the systemic administration comprises enteral administration or parenteral administration.

8-9. (canceled)

10. The method of claim 7, wherein the quantity of TS administered in a 24 hour period ranges from 10 ng to 1 g.

11. The method of claim 7,wherein the quantity of GV administered in a 24 hour period ranges from 10 ng to 1 g.

12. The method of claim 7, wherein the quantity of TLK-199 administered in a 24 hour period ranges from 10 ng to 1 g.

13. The method of claim 1, wherein the contacting comprises contacting with one or more compositions comprising one or more of TS, GV, and TLK-199, optionally wherein the one or more composition further comprise one or more of a pharmaceutically acceptable carrier and excipient.

14. The method of claim 13, wherein the molar concentration of the TS in the composition is from 10 nM to 500 μM.

15. The method of claim 13, wherein the molar concentration of the GV in the composition is from 10 nM to 500 μM.

16. The method of claim 13, wherein the molar concentration of the TLK-199 in the composition is from 10 nM to 500 μM.

17-19. (canceled)

20. The method of claim 7, wherein the subject is a mammal, and optionally is a human.

21. The method of claim 20, wherein the malignant cells are located in the subject in one or more of: the pleura of the lungs, the peritoneum, the pericardium, and the tunica vaginalis.

22. The method of claim 7, wherein the administration comprises a primary treatment provided to the subject, and optionally the subject additionally is administered one or more of a surgical regimen, a radio-therapy regimen, and a chemotherapy regimen.

23. (canceled)

24. The method of claim 1, wherein the malignant cell is a cancer cell.

25. A composition comprising Thiostrepton (TS) and TLK-199 and optionally further comprising gentian violet (GV).

26. (canceled)

27. The composition of claim 25, wherein the molar concentration of the TS in the composition is from 10 nM to 500 optionally the molar concentration of the GV in the composition is from 10 nM to 500 and optionally the molar concentration of the TLK-199 in the composition is from 10 nM to 500 μM.

28-29. (canceled)

30. The composition of claim 25, further comprising one or more of a pharmaceutically acceptable carrier and excipient.
